(19) United States
(12) Patent Application Publication
(10) Pub. No.: US 2017/0224863 A1
(43) Pub. Date: Aug. 10, 2017

(54) WINE CORK POTPOURRI DISPLAY

(71) Applicant: Matt DuBow, Newport Beach, CA (US)

(72) Inventor: Matt DuBow, Newport Beach, CA (US)

(21) Appl. No.: 15/040,937

(22) Filed: Feb. 10, 2016

(51) Int. Cl. A61L 9/12; A47G 35/00

(52) U.S. Cl. CPC A61L 9/12; A47G 35/00; A61L 2209/131

(57) ABSTRACT

A cork potpourri display of randomly oriented scented corks, within a transparent packaging having a vent to allow the scent to escape the packaging. The corks contain markings from their source, presenting an attractive display while also serving to provide a pleasant smell.

### FIELD

This invention relates to potpourri. More particularly, it relates to potpourri composed of “wine” corks that are scented and decoratively packaged.

### BACKGROUND

Potpourri is historically believed to have originated from persons storing cut flowers or fruit rinds, to provide a pleasant odor to a room, while also enhancing its appearance. Potpourri has evolved to many forms, sea shells, shaved/colored woods, etc., the common denominator being the potpourri is visually appealing and also pleasant smelling. However, despite the long history and wide variety of types of potpourri, there has not yet been a potpourri type using scented wine or bottle corks. Moreover, there has not been a wine/bottle cork potpourri container that elegantly and simply captures a display of scented wine/bottle corks, as further described below.

### SUMMARY

The following presents a simplified summary in order to provide a basic understanding of some aspects of the claimed subject matter. This summary is not an extensive overview, and is not intended to identify key/critical elements or to delineate the scope of the claimed subject matter. Its purpose is to present some concepts in a simplified form as a prelude to the more detailed description that is presented later.

In one aspect of the disclosed embodiments, a cork potpourri display is provided, comprising, a substantially transparent packaging adapted to contain an assemblage of marked bottle corks, having an openable vent to allow scents from an interior of the packaging to escape; and an assemblage of bottle corks with markings thereupon randomly oriented in the packaging, treated with a pleasant scent, wherein the marked corks are externally visible through the packaging and scents from the corks are escape through the vent.

In another aspect of the disclosed embodiments, the above display is provided, wherein the packaging is substantially rectangular and less than 1 foot in width and height; and/or wherein the packaging is made of plastic; and/or wherein the vent is at a top and/or lower portion of the packaging; and/or wherein the vent is formed from folds at an end of the packaging; and/or wherein the vent is a plurality of holes; and/or wherein the scent is at least one of vanilla, wine, pomegranate, sage citrus, honeysuckle, spice, citrus cilantro, pumpkin, lavender, spiced apple; and/or wherein the corks are from wine bottles; and/or wherein the corks are from used wine bottles; and/or wherein the vent is covered with a netting or fibrous material; and/or wherein the packaging has a circular form; and/or wherein the corks are synthetic.

In yet another aspect of the disclosed embodiments, a method of making a cork potpourri display is provided, comprising, collecting bottle corks with markings; applying a scent to the corks; and packaging scented corks randomly into a substantially transparent packaging having an openable vent to allow scents from an interior of the packaging to escape, wherein the marked corks are externally visible through the packaging and scents from the corks are escape through the vent.

In yet another aspect of the disclosed embodiments, the above method is provided, wherein the scent is at least one of vanilla, wine, pomegranate, sage citrus, honeysuckle, spice, citrus cilantro, pumpkin, lavender, spiced apple; and/or wherein the corks are from wine bottles; and/or wherein the corks are from used wine bottles; and/or wherein the vent is formed from folds at an end of the packaging.

### BRIEF DESCRIPTION OF THE DRAWINGS

FIG. 1 is a flow diagram showing a cork-to-display process.

FIG. 2 is a diagram of a front view of a cork-potpourri display package.

FIG. 3 is a diagram of a side view of a cork-potpourri display package.

FIG. 4 is a diagram of an end view of a cork-potpourri display package.

FIG. 5 is a diagram of an end view of an opened cork-potpourri display package.

### DETAILED DESCRIPTION

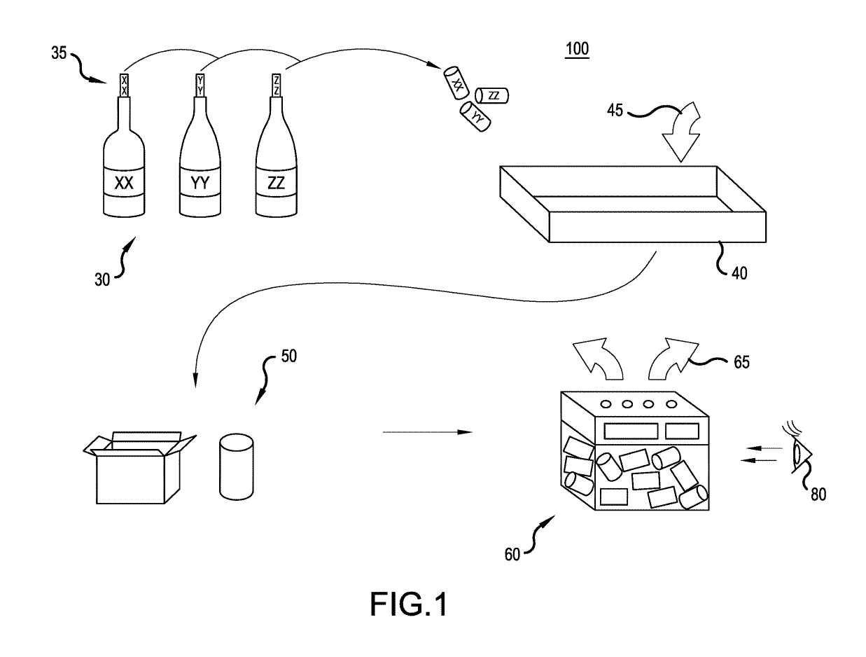

FIG. 1 is a flow diagram 100 showing a cork-to-display process, resulting in the exemplary display. Various assorted bottles 30 having bottle or winery-specific corks 35 are assembled and the corks 35 are removed and placed into a container 40, having a scented chemical or liquid 45 applied thereto. The container 40 may be an open chamber or a closed chamber, or a plurality of chambers. Further the added chemical 45 may be a gas or a solid material that infuses its smells to the corks 35. In some embodiments, the chemical 45 may be a natural product, such as a spice, oil, etc. For example, in some embodiments, the chemical/substance 45 will imbue the scent of any one or more of vanilla, wine, pomegranate, sage citrus, honeysuckle, spice, citrus cilantro, pumpkin, lavender, spiced apple. The corks 35 can be obtained from spent bottles 30 or directly from a bottle/wine cork manufacturer, thus the corks 35 can be in unused, virgin form, if so desired. However, in most embodiments, it is recognized that “used” corks 35 generally have characteristics of the bottle's wine/liquid, including colorations that add to the “character” of the corks 35. In some instances, the corks 35 will undergo a quality control review either prior to placement into container 45 or prior to packaging, as some corks 35 may be damaged or unsuitable for aesthetic display.

It is understood that the corks 35, being associated with a bottle (or winery, or manufacturer of the contents of the bottle) will be marked with the bottle (winery or manufacturer of the bottled good) logo (shown here as using generic letters XX, YY, or ZZ) or some decorative feature. While bottle corks can be natural or synthetic, the benefit for synthetic corks is that they are cheaper, however, they may not absorb the chemicals as readily as natural corks. Also, natural corks are imbued with natural wood traits having an intrinsic aesthetic, the goods-manufacturer's logo or decorative feature imprinted onto the cork is understood to increase the aesthetics of the cork. Thus, a significant and unavoidable visual characteristic of the wine/bottle cork is the imprinted decorative feature. Since different good manufacturers have their own signature decorative features, having an assortment of different corks from different manufacturers provides a unique visual appeal. Of course, in some instances, some manufacturers, being so diversified, will have a sufficient assortment of different variety of products in their line to warrant corks from them only, if so desired.

From the scent imbuing container 40, the scented corks are placed 50 into substantially transparent packaging, shown here as a box and/or cylinder composed of a material that is firm enough to support the corks, non-limiting examples being clear plastic, acrylic, and so forth. Other shapes may be used according to design preference. The important aspects of the packaging 60 is that the bulk of the randomly situated corks can be visible to a customer 80 and the packaging 60 has some mechanism for dispensing 65 the scent from the corks. Further, since the product is ultimately used for potpourri, the packaging 60 is sized for convenient display and shipping to customers, for example, ranging anywhere between 3 to 12 inches in height and 3 to 12 inches in width. Of course, other sizes can be utilized, however, overly large (e.g., 2+ feet large displays) may be counter to the purpose of potpourri. In the image presented here, packaging 60 is shown with a plurality of upper holes, however, other forms of venting and/or locations to allow the scent(s) to escape are within the purview of one of ordinary skill and thus are understood to be within the scope of this disclosure.

The unique visual appeal of “imprinted” wine/bottle corks is known in the decorative industry. For example, the use of corks being arranged to form a cork-bulletin board is known, providing a “functional” use of the corks. Flooring from bottle cork pieces is known, however, all of the prior art are directed to “functional” uses of the cork material itself for a tactile experience. To date, no one has contemplated using “imprinted” bottle corks infused with a scent and packaged to provide both a decorative and olfactory experience, specifically tailored for the potpourri industry.

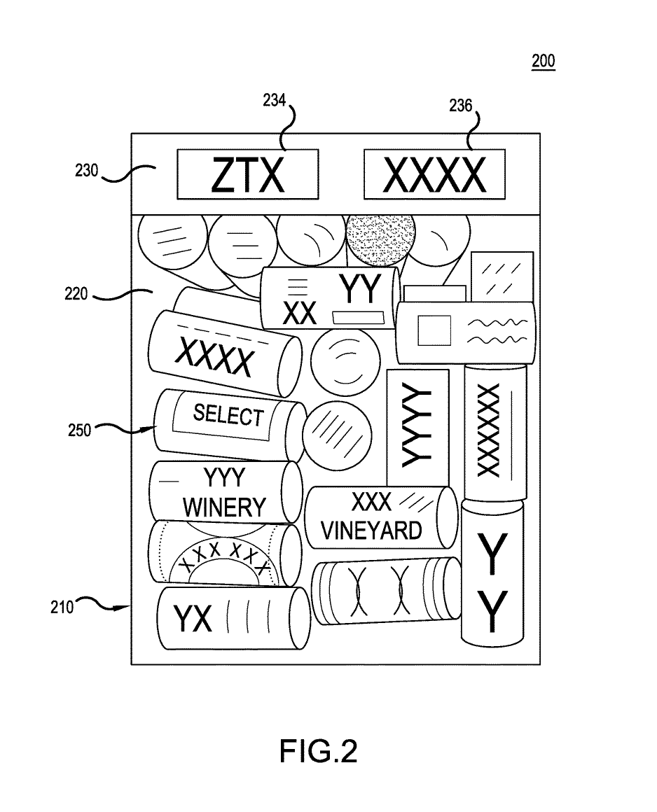

FIG. 2 is a diagram 200 of a front view of an exemplary cork-potpourri display package 210, having a rectangular transparent face 220 allowing viewing of various imprinted corks 250 that are randomly arranged. A portion 230 of the package 210 is reserved for product labeling 234, 236 of the good. While the portion 230 is shown on the upper side of the package 210, it may be placed at any location on the package 210 as well be of any shape desired. Further, while two labels 234, 236 are shown, a single label, multiple labels, other shape(s), size(s), location(s) are contemplated, understanding that labeling and “marking” of a good for display of the manufacturer of the good is a very diverse and varied field. The import of FIG. 2 is to shown key aspects of the packaging 201, namely to allow the viewability of the corks 250 and provide some labeling.

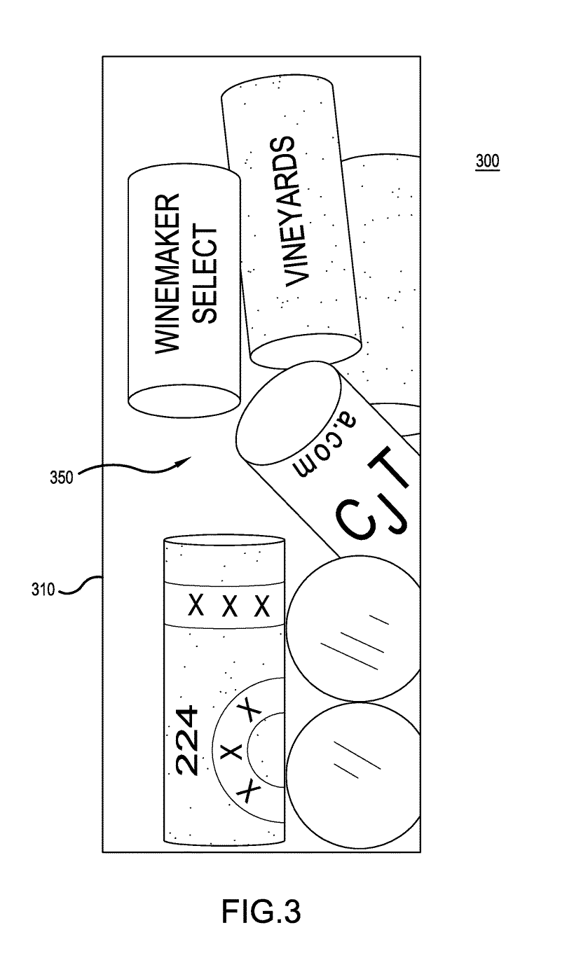

FIG. 3 is a diagram 300 of a side view of a cork-potpourri display package 310 also displaying corks 350. This Fig. is understood to be self explanatory.

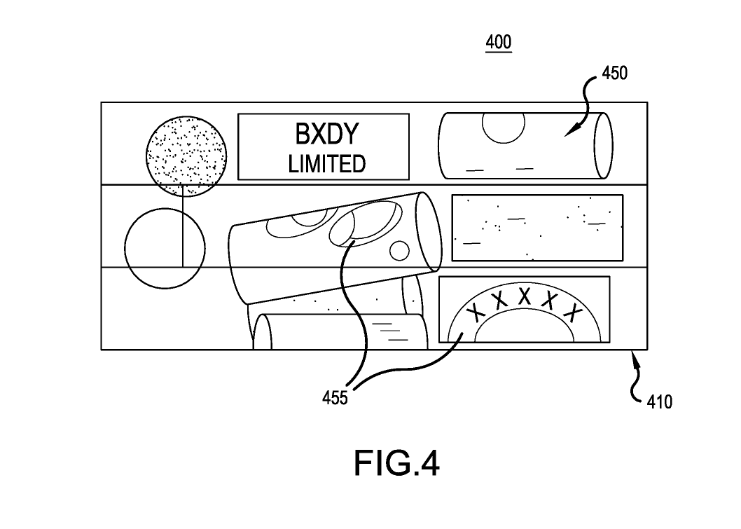

FIG. 4 is a diagram 400 of an end view of a cork-potpourri display package 410 with corks 450 viewable therein. Various designs of the corks' bottle/winery are noted by element 455. This Fig. is understood to be self explanatory.

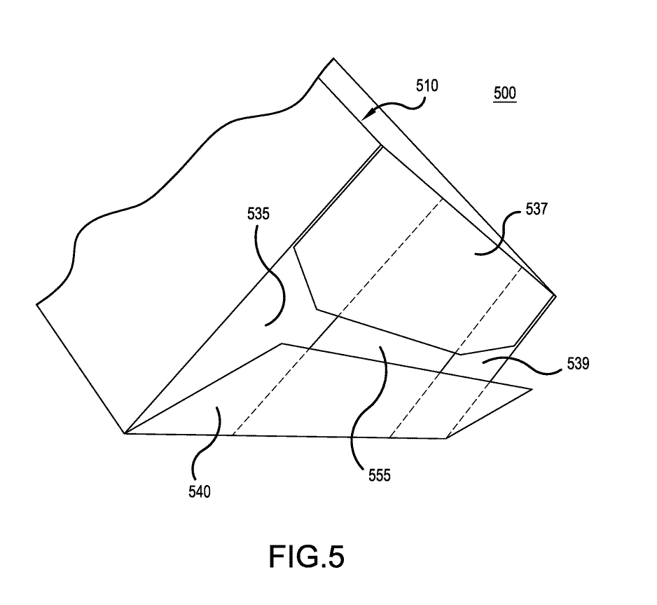

FIG. 5 is a diagram 500 of a perspective end view of a cork-potpourri display package 510 with an “opening” 555 to allow the scents from the corks (not displayed) to escape. Since this embodiment utilizes a box-shaped package 510, the end contains folds 535, 537, 539, and 540 that in one form or another do not span the entirety of the end, thus naturally forming the opening 555. In this embodiment, the opening 555 is rectangular, however other shapes are contemplated, depending on the shaping/arrangement of the folds 535, 537, 539, and 540. Of course, more or less folds may be used without departing from the spirit and scope of this disclosure.

Also, while FIG. 5 show the opening as being formed from “truncated” folds 535, 537, 539, and 540, it is understood that the intent of FIG. 5 is to show one of many various possible ways to provide a venting opening(s) for the scents to escape package 510. Therefore, other openings such as holes, cuts, and so forth are understood to be design changes that are within the purview of one of ordinary skill. For example, opening 555 may be situated at a top or bottom portion of the package 510, rather than at its end. Also, opening 555 may span various other sections of the package 510 (a side or middle). In all the examples, opening 555 is designed to allow the scent to escape but also prevent the corks from fall out of package 510. If the opening is disposed at the upper end of package 510, then the opening 555 may be sized larger than the corks, since is understood that package 510 is maintained in a stationary position. It should be understood that the opening 555 is “closed” during shipping so as to prevent exhaustion of the corks' smells. Thus, the customer can “open” the vent upon use, either by removing a sealing membrane or breaking open a seal. The opening procedure, in some embodiments, can simply be opening the “top” of the package 510.

If opening 555 is made larger than the corks, then it is contemplated that a mesh or other restricting element can be implemented to retain the corks. In some embodiments, the entire/majority of package 510 or a section of package 510 may be made of a firm mesh, providing structural support, viewability of the corks, while scents to escape. Further, since the opening 555 purpose is to provide a venting opening for the scents, it is contemplated that some “solid” materials that provide gas permeability can be used, as a proxy to the opening 555, such as netting or fibrous sheet, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope being indicated by the following claims.

What is claimed is:

1. A cork potpourri display, comprising:
a substantially transparent packaging adapted to contain an assemblage of marked bottle corks, having an openable vent to allow scents from an interior of the packaging to escape; and
an assemblage of bottle corks with markings thereupon randomly oriented in the packaging, treated with a pleasant scent,
wherein the marked corks are externally visible through the packaging and scents from the corks are escape through the vent.

2. The display of claim 1, wherein the packaging is substantially rectangular and less than 1 foot in width and height.

3. The display of claim 1, wherein the packaging is made of plastic.

4. The display of claim 1, wherein the vent is at a top and/or lower portion of the packaging.

5. The display of claim 4, wherein the vent is formed from folds at an end of the packaging.

6. The display of claim 1, wherein the vent is a plurality of holes. The display of claim 1, wherein the scent is at least one of vanilla, wine, pomegranate, sage citrus, honeysuckle, spice, citrus cilantro, pumpkin, lavender, spiced apple.

8. The display of claim 1, wherein the corks are from wine bottles.

9. The display of claim 8, wherein the corks are from used wine bottles.

10. The display of claim 1, wherein the vent is covered with a netting or fibrous material.

11. The display of claim 1, wherein the packaging has a circular form.

12. The display of claim 1, wherein the corks are synthetic.

13. A method of making a cork potpourri display, comprising:
collecting bottle corks with markings;
applying a scent to the corks; and
packaging scented corks randomly into a substantially transparent packaging having an openable vent to allow scents from an interior of the packaging to escape,
wherein the marked corks are externally visible through the packaging and scents from the corks are escape through the vent.

14. The method of claim 13, wherein the scent is at least one of vanilla, wine, pomegranate, sage citrus, honeysuckle, spice, citrus cilantro, pumpkin, lavender, spiced apple.

15. The method of claim 13, wherein the corks are from wine bottles.

16. The method of claim 15, wherein the corks are from used wine bottles.

17. The method of claim 13, wherein the vent is formed from folds at an end of the packaging.
